(19) United States
(12) Patent Application Publication
(10) Pub. No.: US 2012/0063952 A1
(43) Pub. Date: Mar. 15, 2012

(54) UV RESISTANT CLEAR LAMINATES

(76) Inventors: Keith C. Hong, Lititz, PA (US); Frank J. Csillag, Hopkinton, MA (US)

(21) Appl. No.: 13/212,328

(22) Filed: Aug. 18, 2011

Related U.S. Application Data
(60) Provisional application No. 61/381,574, filed on Sep. 10, 2010.

(51) Int. Cl. A61L 2/10; B05D 5/10; B32B 27/08

(52) U.S. Cl. 422/24; 428/421; 427/207.1

(57) ABSTRACT

A multilayer construct includes a fluoropolymer first layer; a UV resistant fluoropolymer adhesive layer, and a third layer, wherein the fluoropolymer adhesive layer is between the first and third layers. The adhesive layer can optionally include an ultraviolet radiation absorber.

### CROSS REFERENCE TO RELATED APPLICATIONS

This application claims benefit under 35 U.S.C. §119(e) to U.S. Provisional Patent Application Ser. No. 61/381,574, filed Sep. 10, 2010, the contents of which is incorporated herein by reference in its entirety.

### FIELD OF THE DISCLOSURE

This disclosure, in general, relates to multilayer constructs and electronic and photovoltaic devices formed there from.

### BACKGROUND

With increasing energy prices and with increasing concern over the environmental impact of hydrocarbon fuels, industry is turning to alternative energy sources, such as solar power. In particular, industry is turning to photovoltaic devices which convert sunlight into electrical current. Although photovoltaic devices represent low ongoing operational costs, much of the expense of installing a photovoltaic device is in upfront equipment costs. As such, economic viability of a photovoltaic device is strongly dependent upon equipment cost and durability.

During use, photovoltaic devices are exposed to ultraviolet light. To protect the photovoltaic devices, encapsulants and other polymer films are disposed over the surfaces of the photovoltaic devices. However, such encapsulants and other polymer films are themselves susceptible to ultraviolet light and over time may degrade. Such degradation reduces the effectiveness of encapsulants and polymer films, leading to possible damage to the photovoltaic devices or reduced operational usefulness of the device due to degradation of the protective film.

Durability concerns influence the competitiveness of photovoltaic systems relative to other energy sources. Despite the attractiveness of the low environmental impact of solar energy solutions, photovoltaic devices are struggling to provide electricity at existing grid prices. A reduction in durability severely hampers the viability of existing photovoltaic operations.

As such, an improved photovoltaic system would be desirable.

### BRIEF SUMMARY OF THE INVENTION

The present invention pertains to a multilayer construct, such as a film, that includes a fluoropolymer first layer; a UV resistant fluoropolymer adhesive layer disposed upon the fluoropolymer first layer or a third layer; and a third layer disposed upon the fluoropolymer adhesive layer or to which the fluoropolymer layer adhesive was disposed upon.

In another embodiment the invention pertains to a construct that includes a fluoropolymer first layer and a UV resistant fluoropolymer adhesive layer disposed upon the first layer.

In certain aspects, the UV resistant fluoropolymer adhesive layer can react to external conditions to cause crosslinking, condensation, or other reactions to occur. Thus the UV resistant fluoropolymer adhesive can be considered a “reactive” bonding layer. The UV resistant fluoropolymer layer can react with an additive, such as a crosslinking agent, or can react with functional moieties within the adhesive itself.

The UV resistant fluoropolymer adhesive layer is inherently UV resistant due to the chemical/physical nature of the polymer. One advantage of the UV resistant fluoropolymer layer is that a UV absorbing material does not need to be added to the adhesive to achieve UV resistant properties. Thus, the interface between the UV resistant fluoropolymer adhesive and the contact layers is superior to adhesives that contain UV absorbing additives which may reduce the adhesiveness of the material. Additionally, the UV resistant fluoropolymer adhesive in terms of UV resistance may provide more uniform UV resistance throughout the multilayer film in comparison to a system where a UV absorbing material is added which may be more heterogeneous in nature.

Typically the fluoropolymer is selected from poly vinylidene fluoride (PVDF), polyvinyl fluoride (PVF), polytetrafluoroethylene (PTFE), a copolymer of tetrafluoroethylene and perfluoro methylvinylether (PFA), ethylene tetrafluoroethylene copolymer (ETFE), polychlorotrifluoroethylene (PCTFE), ethylene chlorotrifluoroethylene copolymer (ECTFE), fluorinated ethylene propylene copolymer (FEP), a copolymer of ethylene and fluorinated ethylene propylene (EFEP), a terpolymer of tetrafluoroethylene, hexafluoropropylene, and vinylidene fluoride (THV), a terpolymer of tetrafluoroethylene, hexafluoropropylene, and ethylene (HTE), or any combination thereof.

In various embodiments, the third layer can be any material that can adhere to the UV resistant fluoropolymer adhesive.

### BRIEF DESCRIPTION OF THE DRAWINGS

The present disclosure may be better understood, and its numerous features and advantages made apparent to those skilled in the art by referencing the accompanying drawings.

FIG. 1 is a drawing of a multilayer construct that includes a fluoropolymer layer, a UV resistant fluoropolymer adhesive and a third layer.

The use of the same reference symbols in different drawings indicates similar or identical items.

### DESCRIPTION OF THE PREFERRED EMBODIMENT(S)

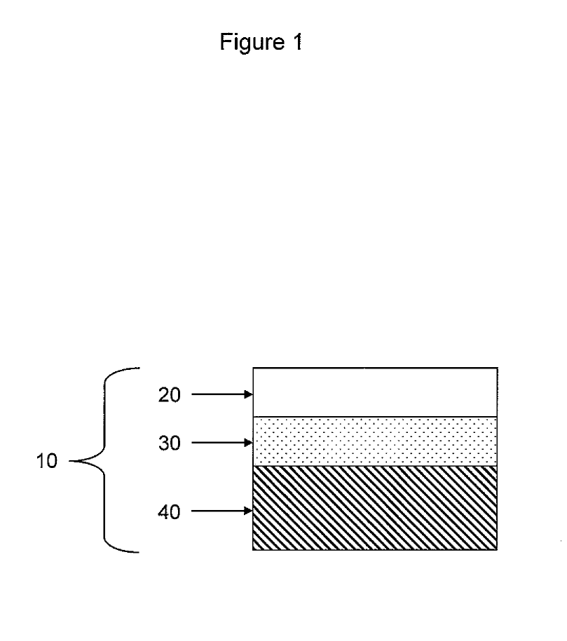

As illustrated in FIG. 1, a multilayer construct 10 of the present invention includes a fluoropolymer layer 20, a UV resistant fluoropolymer adhesive layer 30 and a third layer, such as a protective layer 40.

It should be understood that the term “multilayer construct” includes multilayer films. Typically a multilayer construct is thicker than a multilayer film. Multilayer film thicknesses typically range from about 1 mils to about 30. A construct thickness can vary greatly where the protective, or third layer, 40 can be from about 0.5 mils to about 100 mils and even thicker. For example, fluoropolymer layer 20 and UV resistant fluoropolymer adhesive layer 30 can be applied to a brick to provide a multilayer construct. It should be understood that where the terms “multilayer film” and “multilayer construct” are used interchangeably throughout the specification and are not limiting.

In one embodiment, multilayer construct 10 is a multilayer film. Multilayer film 10 includes fluoropolymer layer 20 and UV resistant fluoropolymer adhesive layer 30. This construct can then be applied to a third layer or substrate at a later time. Suitable third layers/substrates are noted herein.

In another embodiment, multilayer construct 10 is also multilayer film. Multilayer film 10 can be used as a barrier or protective layer for a photovoltaic device. The multilayer film 10 can also be used as a barrier or protective layer for any article requiring durable, transparent protection in an outdoor environment. Such articles may include out door signage, awnings, roofing, outdoor electronic devices and the like. One advantage of the multilayer films of the invention is to allow visible light through the film while maintaining the adhesive bond between fluoropolymer layer 20 and/or layer 40 without requiring the use of added UV absorbers. In an alternative embodiment, UV absorbers can be added to the adhesive layer to protect the layer(s) below. When layer 40 is either inherently UV resistant, such as a fluoropolymer, or alternatively pre-loaded with UV additives, such as DuPont Teijin XST6638 or X6641 PET film, a UV absorber would not be required in the adhesive layer. Not to be limited by theory, it is believed that use of UV resistant fluoropolymer adhesive layer materials that are inherently UV resistant (not requiring a UV absorber as an additive) helps promote improved adhesion between the various layers.

Fluoropolymer layer 20 has a thickness in a range of 0.5 mils to 20 mils. For example, layer 20 can have a thickness in a range of 0.5 mils to 10 mils, such as a range of 0.5 mils to 5 mils, or even 0.5 mils to 2 mils.

UV resistant fluoropolymer adhesive layer 30 underlies layer 20. In an example, the UV resistant fluoropolymer adhesive layer 30 is in direct contact with layer 20 without intervening layers.

UV resistant fluoropolymer adhesive layer 30 can have a thickness in a range of 0.2 mils to 10 mils, such as a range of 0.2 mils to 5 mils, 0.2 mils to 2 mils, such as a range of 0.2 mils to 1.5 mils, or a range of 0.5 mils to 1.0 mils. Alternatively, UV resistant fluoropolymer adhesive layer 30 can have a thickness in a range of 2 mils to 10 mils.

Exposure to sunlight and other artificial lights can have adverse effects on the useful life of plastic products, such as multilayer films. UV radiation can break down the chemical bonds in a polymer. This process is called photodegradation and ultimately causes cracking, chalking, color changes and the loss of physical properties.

Up until this invention, UV stabilizers were added to materials to solve the degradation problems associated with exposure to sunlight as one approach to counteract the damaging effect of UV light. UV stabilizers can be categorized by two general classifications—ultraviolet light absorber (UVA) and hindered amine light stabilizers (HALS).

Ultraviolet absorbers function by preferentially absorbing harmful ultraviolet radiation and dissipating it as thermal energy. The commonly available UV absorbers are based on benzophenones, benzotriazoles, triazines, or benzoxazinones.

Multilayer films of the invention can be prepared by coating or by lamination. In one embodiment, the multilayer film includes a fluoropolymer, UV resistant fluoropolymer adhesive and a third layer. In another embodiment, the multilayer film can be adhered to a photovoltaic device.

It should be understood that the multilayer films of the invention can include from 2 layers to about 12 layers of material. For example, the multilayer films can repeat layering of a first layer and a second layer, and so forth. Additionally, combinations of various layers are included herein, for example, a first layer, a second layer, a third layer differing from the first or second layers and a fourth layer which differs from the first, second or third layers, etc. This layering, again, can be repeated as needed for the application envisioned.

In one aspect, a two layer construct of a fluoropolymer and a UV resistant fluoropolymer adhesive can be vacuum sealed in a plastic container, such as a bag, to limit film exposure to moisture. The construct can then later be applied to a third layer or substrate and adhered to the third layer or substrate via exposure to atmospheric moisture.

Among the classes of polymers, fluoropolymers are unique materials because they exhibit an outstanding range of properties such as high transparency, good dielectric strength, high purity, chemical inertness, low coefficient of friction, high thermal stability, excellent weathering, and UV resistance. Fluoropolymers are frequently used in applications calling for high performance in which oftentimes the combination of the above properties is required.

The first layer of the multilayer construct (20) is generally a fluoropolymer. The phrase “fluoropolymer” is known in the art and is intended to include, for example, polytetrafluoroethylene, copolymers of tetrafluoroethylene and hexafluoropropylene, tetrafluoroethylene-perfluoro(alkyl vinyl ether) copolymers (e.g., tetrafluoroethylene-perfluoro(propyl vinyl ether), FEP (fluorinated ethylene propylene copolymers), polyvinyl fluoride, polyvinylidene fluoride, and copolymers of vinyl fluoride, chlorotrifluoroethylene, and/or vinylidene fluoride (i.e., VDF) with one or more ethylenically unsaturated monomers such as alkenes (e.g., ethylene, propylene, butylene, and 1-octene), chloroalkenes (e.g., vinyl chloride and tetrachloroethylene), chlorofluoroalkenes (e.g., chlorotrifluoroethylene, 3-chloropentafluoropropene, dichlorodifluoroethylene, and 1,1-dichlorofluoroethylene), fluoroalkenes (e.g., trifluoroethylene, tetrafluoroethylene (i.e., TFE), 1-hydropentafluoropropene, 2-hydropentafluoropropene, hexafluoropropylene (i.e. HFP), and vinyl fluoride), perfluoroalkoxyalkyl vinyl ethers (e.g., CF₃OCF₂CF₂CF₂OCF═CF₂); perfluoroalkyl vinyl ethers (e.g., CF₃OCF═CF₂ and CF₃C₂CF₂OCF═CF₂) and combinations thereof.

The fluoropolymer can be melt-processable, for example, as in the case of polyvinylidene fluoride; copolymers of vinylidene fluoride; copolymers of tetrafluoroethylene, hexafluoropropylene, and vinylidene fluoride (e.g., those marketed by Dyneon, LLC under the trade designation “THV”); copolymers of tetrafluoroethylene and hexafluoropropylene; and other melt-processable fluoroplastics; or the fluoropolymer may not be melt-processable, for example, as in the case of polytetrafluoroethylene, copolymers of TFE and low levels of fluorinated vinyl ethers), and cured fluoroelastomers.

Useful fluoropolymers include those copolymers having HFP and VDF monomeric units.

Useful fluoropolymers also include copolymers of HFP, TFE, and VDF (i.e., THV), such as those available from Dyneon, LLC.

Other useful fluoropolymers also include copolymers of ethylene, TFE, and HFP, also available from Dyneon, LLC.

Additional commercially available vinylidene fluoride-containing fluoropolymers include, for example, those fluoropolymers having the trade designations; “KYNAR” (e.g., “KYNAR 740”) as marketed by Arkema; “HYLAR” (e.g., “HYLAR 700”) as marketed by Solvay Solexis and “FLUOREL” (e.g., “FLUOREL FC-2178”) as marketed by Dyneon, LLC. Copolymers of vinylidene fluoride and hexafluoropropylene are also useful. These include for example KYNARFLEX (e.g. KYNARFLEX 2800 or KYNARFLEX 2550) as marketed by Arkema.

Commercially available vinyl fluoride fluoropolymers include, for example, those homopolymers of vinyl fluoride marketed under the trade designation “TEDLAR” by E.I. du Pont de Nemours & Company, Wilmington, Del.

Useful fluoropolymers also include copolymers of ethylene and TFE (i.e., “ETFE”) available from DuPont, Daikin, Dyneon, Ashai and others.

Additionally, useful fluoropolymers include copolymers of ethylene and chlorotrifluoroethylene (ECTFE). Commercial examples include Halar 350 and Halar 500 resin from Solvay Solexis Corp.

Other useful fluoropolymers include substantially homopolymers of chlorotrifluoroethylene (PCTFE) such as Aclar from Honeywell.

In another aspect, the fluoropolymer layer can be surface treated. Generally, hydrophilic functionalities are attached to the fluoropolymer surface, rendering it easier to wet and provides opportunities for chemical bonding. There are several methods to functionalize a fluoropolymer surface including plasma etch, corona treatment, chemical vapor deposition, or any combination thereof. In another embodiment, plasma etching includes reactive plasmas such as hydrogen, oxygen, acetylene, methane, and mixtures thereof with nitrogen, argon, and helium. Corona treatment can be conducted in the presence of reactive hydrocarbon vapors such as ketones, e.g., acetone, C1-C4 carbon chain length alcohols, p-chlorostyrene, acrylonitrile, propylene diamine, anhydrous ammonia, styrene sulfonic acid, carbon tetrachloride, tetraethylene pentamine, cyclohexyl amine, tetra isopropyl titanate, decyl amine, tetrahydrofuran, diethylene triamine, tertiary butyl amine, ethylene diamine, toluene-2,4-diisocyanate, glycidyl methacrylate, triethylene tetramine, hexane, triethyl amine, methyl alcohol, vinyl acetate, methylisopropyl amine, vinyl butyl ether, methyl methacrylate, 2-vinyl pyrrolidone, methylvinylketone, xylene or mixtures thereof.

Some techniques use a combination of steps including one of these methods. For example, surface activation can be accomplished by plasma or corona in the presence of an excited gas species.

Not to be limited by theory, the method has been found to provide strong interlayer adhesion between a modified fluoropolymer and an adhered layer. The fluoropolymer can be surface treated by the process described in U.S. Pat. Nos. 3,030,290; 3,255,099; 3,274,089; 3,274,090; 3,274,091; 3,275,540; 3,284,331; 3,291,712; 3,296,011; 3,391,314; 3,397,132; 3,485,734; 3,507,763; 3,676,181; 4,549,921; and 6,726,979, the teachings of which are incorporated herein in their entirety for all purposes.

In one aspect, the surface of the fluoropolymer substrate is treated with a corona discharge where the electrode area was flooded with acetone, a C1-C4 carbon chain length alcohol, tetrahydrofuran methyl ethyl ketone, ethyl acetate, isopropyl acetate or propyl acetate vapors.

Corona discharge is produced by capacitative exchange of a gaseous medium which is present between two spaced electrodes, at least one of which is insulated from the gaseous medium by a dielectric barrier. It is a high voltage, low current phenomenon with voltages being typically measured in kilovolts and currents being typically measured in milliamperes. Corona discharges may be maintained over wide ranges of pressure and frequency. Pressures of from 0.2 to 10 atmospheres generally define the limits of corona discharge operation and atmospheric pressures generally are desirable. Frequencies ranging from 20 Hz to 100 kHz can conveniently be used: in particular ranges are from 500 Hz, especially 3000 Hz to 10 kHz.

In another aspect, the surface of the fluoropolymer is treated with a plasma. The plasma is generally created by RF (AC) frequency or DC discharge between two electrodes where in between the substrate is placed and the space is filled with the reacting gases. A plasma is any gas in which a significant percentage of the atoms or molecules are ionized, resulting in reactive ions, electrons, radicals and UV radiation.

UV resistant fluoropolymer adhesive layer 30 useful in the multilayer constructs described herein are fluoropolymers that have been chemically modified to contain reactive functional groups along the backbone or side chains. Additionally, other chemical modifications can provide the fluoropolymers with properties such as solubility in common organic solvents and increased flexibility. One cross-linkable fluoropolymer adhesive is the Zeffle® adhesive available from Daikin. Zeffle® is a copolymer of tetrafluoroethylene and olefins with reactive hydroxyl groups. Another cross-linkable fluoropolymer adhesive is Lumiflon® adhesive from Asahi Glass. Lumiflon® is a chlorotrifluoroethylene polymer with alkyl vinyl ethers and hydroxyl groups. These modified fluoropolymer adhesives can be reacted with a whole array of chemical crosslinking agent such as aliphatic isocyanates, titanates and melamines. The chemical reactions result in excellent adhesion and also renders the adhesive insoluble for long term durability.

In one aspect, the UV resistant fluoropolymer adhesive is functionalized with one or more moieties selected from an olefinic moiety, such as an unsaturated olefin containing funcational group, a hydroxyl, an ether moiety, a methacrylate, an acrylate, an epoxide, a silane moiety, a phosphoric acid moiety, a sulfonic acid moiety or a carboxylic acid moiety.

In another aspect, the fluoropolymer adhesive is a polyperfluoroalkyl methacrylate or polyperfluoroalkyl acrylate.

In still another aspect, the fluoropolymer adhesive is a perfluorourethane alkyd, a perfluoropolyether, a perfluoro silane, or a perfluoro epoxide such as those sold as FLUOROPEL™, FLUOROTHANE™, or FLUOROSYL™, by Cytonix Corp.

In another aspect, the fluoropolymer adhesive is a functionalized perfluoro-polyether polymer functionalized with an alkyl amide, an alkoxy silane or a phosphate moiety such as those known as FLUOROLINK™ by Solvay Solexis.

In still yet another aspect, the fluoropolymer adhesive is an amorphous fluoropolymer known as TEFLON® AF by DuPont.

A crosslinking agent can be added to the fluoropolymer adhesive. Suitable crosslinking agents include, for example, mono, di and multifunctional agents selected from amines, amides, anhydrides, phenolics, isocyanates, hydroxyls, carboxyls, titanates, zirconates, aluminates, proton donors, peroxides, silanes, metal ions, moisture or combinations thereof.

UV resistant fluoropolymer adhesive 30 is functionalized with moieties that can undergo a reaction with either a crosslinker or can react with functionality contained within the fluoro adhesive material itself. The reaction can be facilitated by heat, pressure, moisture, pH change, UV light, thermal initiators, such as peroxides, persulfates, light sensitive initiators, radiation, such as gamma rays and electron beam, acid catalysts, base catalysts, metal ions or combinations thereof.

UV resistant fluoropolymer adhesive layer 30 can include an additional ultraviolet radiation absorber. In addition, UV resistant fluoropolymer adhesive layer 30 can optionally include a light stabilizer and can optionally include an antioxidant.

In a particular example, UV resistant fluoropolymer adhesive layer 30 bonds to fluoropolymer layer 20, with a peel strength of at least 2 N/cm, such as a peel strength of at least 2.0 N/cm, at least 5 N/cm, at least 10 N/cm, or even at least 15 N/cm. Similarly, adhesive layer 30 bonds to the third layer with similar peel strengths.

In some embodiments, a UV absorber can optionally be added to the UV resistant fluoropolymer adhesive material 30.

UV resistant fluoropolymer adhesive 30 can include the ultraviolet radiation absorber in an amount in a range of 0.1 wt % to 15 wt %, such as a range of 0.1 wt % to 10 wt %. In particular, UV resistant fluoropolymer adhesive 30 can include at least 5.5 wt % of the ultraviolet radiation absorber, such as at least 7.0 wt % of the ultraviolet radiation absorber. In a further example, UV resistant fluoropolymer adhesive 30 does not include greater than 15.0 wt % of the ultraviolet radiation absorber.

Suitable UV absorbers include, for example, benzophenones, such as CYASORB UV-9 (2-hydroxy-4-methoxybenzophenone, CHIMASSORB 81 (or CYASORB UV 531) (2 hydroxy-4 octyloxybenzophenone).

TINUVIN P, TINUVIN 234, TINUVIN 326, TINUVIN 328, CYASORB UV 5411 and CYASORB UV 237 are suitable examples of benzotriazoles.

CYASORB UV 1164 (2-[4,6-bis(2,4-dimethylphenyl)-1,3,5-triazin-2yl]-5(oxctyloxy)phenol is an exemplary triazine UV absorber.

CYASORB 3638 is a suitable UV absorber which is a benzoxazinone.

In addition, UV resistant fluoropolymer adhesive 30 can include a light stabilizer, such as a hindered amine light stabilizer (HALS). Hindered amine light stabilizers (HALS) are extremely efficient stabilizers against light-induced degradation of most polymers. They do not generally absorb UV radiation, but act to inhibit degradation of the polymer. These are typically tetra alkyl piperidines, such as 2,2,6,6-tetramethyl-4-piperidinamine and 2,2,6,6-tetramethyl-4-piperidinol.

For example, UV resistant fluoropolymer adhesive 30 can include the light stabilizer in an amount in a range of 0.1 wt % to 5 wt %. In an example, UV resistant fluoropolymer adhesive 30 includes at least 2.5 wt % of the light stabilizer, such as at least 3.5 wt %, or even at least 5.0 wt % of the light stabilizer. An exemplary light stabilizer is available as Tinuvin® 770 from Ciba Specialty Chemicals or as Cyasorb THT-4611 from Cytech Industries.

The third layer 40 of the multilayer construct, can be for example, natural or synthetic polymers including a fluoropolymer (as described above), polyethylene (including linear low density polyethylene, low density polyethylene, high density polyethylene, etc.), polypropylene, nylons (polyamides), EPDM, polyesters, polycarbonates, ethylene-propylene elastomer copolymers, copolymers of ethylene or propylene with acrylic or methacrylic acids, acrylates, methacrylates, ethylene-propylene copolymers, poly alpha olefin melt adhesives such including, for example, ethylene vinyl acetate (EVA), ethylene butyl acrylate (EBA) ethylene methyl acrylate (EMA); ionomers (acid functionalized polyolefins generally neutralized as a metal salt), acid functionalized polyolefins, polyurethanes including, for example, thermoplastic polyurethane (TPU), olefin elastomers, olefinic block copolymers, thermoplastic silicones, polyvinyl butyral, a fluoropolymer, such as a terpolymer of tetrafluoroethylene, hexafluoropropylene, vinylidene fluoride, metal, composites, glass, cementitious materials, stone, wood or any other suitable substrates or any combination thereof.

Third layer 40 generally has a thickness in a range of 0.5 mils to 20 mils. For example, layer 40 can have a thickness in a range of 0.5 mils to 10 mils, such as a range of 0.5 mils to 5 mils, or even 0.5 mils to 2 mils. However, layer 40 can have much greater thickness when a multilayer construct is provided.

Layer 40 can include an inorganic layer deposited on the surface of the layer. For example, the inorganic layer can include metal, metal oxide, metal nitride, metal carbide, or a combination thereof. In an example, the metal can include aluminum, silver, gold, titanium, tin, zinc, or a combination thereof. An exemplary metal oxide can include alumina, silica, tin oxide, zinc oxide, or a combination thereof. An exemplary metal nitride can include aluminum nitride, titanium nitride, silicon nitride, zinc nitride or a combination thereof. An exemplary carbide can include silicon carbide, aluminum carbide, titanium carbide, or a combination thereof. The thickness of the inorganic layer can be in a range of 30 nm to 1000 nm, such as a range of 50 nm to 500 nm, or even a range of 50 nm to 200 nm.

The multilayer constructs of the invention can be used to protect, in particular, electronic components from moisture, weather, heat, radiation, physical damage and/or insulate the component. Examples of electronic components include, but are not limited to, packaging for crystalline-silicon based thick photovoltaic modules, amorphous silicon, CIGS, CdTe, OPV, or DSSC based thin photovoltaic modules, LEDs, LCDs, printed circuit boards, flexible displays and printed wiring boards.

In one embodiment, the multilayer construct can be laminated to a photovoltaic structure to form the photovoltaic device. For example, a photovoltaic component can be dispensed and a protective construct including a multilayer laminate can be applied over the photovoltaic component. Optionally, an encapsulant can be laminated to the photovoltaic component prior to application of the multilayer protective construct and the multilayer protective construct can be laminated to the encapsulant. Alternatively, a barrier construct can be applied over the photovoltaic component separately from the adhesive and fluoropolymer layers.

For example, the photovoltaic components include at least two major surfaces. The term “front surface” refers to the surface of the photovoltaic device that receives the greater proportion of direct sunlight. In embodiments, the front surface is the active side of the photovoltaic device that converts sunlight to electricity. However, in some embodiments, the photovoltaic device can be constructed such that two surfaces of the device are active. For example, the front surface can convert direct sunlight to electricity, while the back surface can convert reflected sunlight to electricity. In other examples, the front surface can receive direct sunlight at one point during the day and the back surface at another point during the day. The embodiments described herein can include such photovoltaic constructions or other similar photovoltaic constructions. The terms “over,” “overlie,” “under,” or “underlie” refer to the disposition of a layer, construct or laminate relative to a major surface of an adjacent structure in which over or overlie mean the layer, construct or laminate is relatively closer to an outer surface of a photovoltaic device and under or underlie mean the layer, construct or laminate is relatively further from an outer surface of the photovoltaic device. Herein, the terms “on,” “over,” “overlie,” “under,” and “underlie” can permit inclusion of intermediate structures between the surface and the recited structure.

In another embodiment, the multilayer construct of the invention can be formed into a container or a bag suitable for storage of food stuffs such as snack foods including, but not limited to, pretzels, potato chips, crackers, candy and the like.

In another embodiment, the multilayer construct of the invention can be formed into a container or bag suitable for pharmaceutical or medical applications. In a further aspect of this embodiment, the pharmaceutical or medical component contained within the multilayer construct container or bag may be irradiated with UV light for sterilization, when the fluoropolymer adhesive layer is constructed without optional UV absorbers.

Multilayer constructs of this invention can be formed by a variety of methods In the case of a three layer construct to be formed by a sequence of process steps over a short period of time, for example, a few minutes to hours, or even several days, a preferred process is coating of the UV resistant fluoropolymer adhesive on to either the first fluoropolymer layer or the third layer of the construct. Any solvent in the adhesive, if present, is evaporated, and the adhesive layer is then contacted to the remaining layer of the construct. Final adhesion of the three layer construct may be achieved with the pressure with the optional use of heat, in a laminating process, during which time final reaction of the adhesive may be achieved.

In the case of a two layer construct to be adhered to a third layer at a subsequent later time, for example, days, weeks or even months, a preferred process is to coat the adhesive to the first fluoropolymer layer in a form that is only partially cured, or uncured. This partially cured two layer construct may then be suitably packaged, for example with a release liner to inhibit self bonding, and/or with protective sealing to eliminate atmospheric moisture, radiation or any environmental exposure designed to affect subsequent cure. When the two layer construct is later applied to the third layer, cure may then be affected by the selected cure mechanism (exposure to moisture, radiation, additional heat, or the like as described herein).

Typically elevated temperature is used for adhering the multilayers of this invention. Typical temperature ranges are generally at a temperature sufficient to induce crosslinking of the UV resistant fluoropolymer in the case of a crosslinking reaction.

Typically, the lamination of the two heated substrates is conducted under pressure or vacuum. This can be accomplished by many known methods in the art, such as vacuum lamination or roll press lamination. Typical pressure applied to the multilayer construct is about 15 psi to about 45 psi, although it can be higher. When a photovoltaic element is already in contact with the multilayer construct, the pressure is controlled so that the photovoltaic element would not be damaged during processing.

In general, the lamination of the two (or more) substrates is accomplished over a period of from about less than a second to several seconds when a roll press process is utilized. Where vacuum lamination is utilized, the process can take about 5 to about 15 minutes for complete lamination of the two or more materials.

Alternatively, lamination could potentially be done in a roll press. So, for a fluoropolymer construct and a non fluoropolymer contacted in a roll press, low pressure might be from about 1 to about 10 psi, medium pressure could be from about 10 to about 100 psi, high pressure could be from about 100 to about 500 psi, and in extreme cases, even up to about 5000 psi for steel-on-steel nips.

Coating of the layers to each other can be done by conventional methods. For example, the UV resistant fluoropolymer adhesive can be applied to either the first layer or third layer. The remaining layer can then be applied to the layer which has been coated with the adhesive.

Adhesion of the substrates is at least about 2 N/cm. Suitable ranges include up to about 15 N/cm, in particular from about 2 to about 10 N/cm and particularly from about 5 to about 10 N/cm as measure by ASTM D-903 (T-peel test method with a travel speed of 2 inch/min).

The multilayer constructs described herein can have high optical transparencies.

The multilayer constructs of the invention can be used as a front sheet for use with electronic devices such as a photovoltaic device. Alternatively, the multilayer films can be used as a clear backsheet for an electronic device such as a photovoltaic device. It should be understood that the multilayer film can be placed on any active face of an electronic device such as a photovoltaic device.

The present invention also includes “kits”. For example, a multilayer construct of the invention can be packaged in a suitable material or container and sold with instructions for further use, such as application to an electronic device. As another example, where the construct is a two layer construct where the UV resistant fluoropolymer adhesive (with or without a crosslinker or crosslinking agent) is applied to a fluoropolymer film, the construct can be packaged so that it can be applied at a later time to another layer or substrate. Again, instructions can be included that would provide suitable conditions to apply the construct to a third layer such that the construct adheres to the additional layer.

The following paragraphs enumerated consecutively from 1 through 64 provide for various aspects of the present invention. In one embodiment, in a first paragraph (1), the present invention provides a multilayer construct comprising: a fluoropolymer first layer; a UV resistant fluoropolymer adhesive layer, and a third layer, wherein the fluoropolymer adhesive layer is between the first and third layers.

2. The multilayer construct of paragraph 1, wherein the fluoropolymer first layer is selected from poly vinylidene fluoride (PVDF), polyvinyl fluoride (PVF), polytetrafluoroethylene (PTFE), a copolymer of tetrafluoroethylene and perfluoro methylvinylether (PFA), ethylene tetrafluoroethylene copolymer (ETFE), polychlorotrifluoroethylene (PCTFE), ethylene chlorotrifluoroethylene copolymer (ECTFE), fluorinated ethylene propylene copolymer (FEP), a copolymer of ethylene and fluorinated ethylene propylene (EFEP), a terpolymer of tetrafluoroethylene, hexafluoropropylene, and vinylidene fluoride (THV), a terpolymer of tetrafluoroethylene, hexafluoropropylene, and ethylene (HTE), or any combination thereof.

3. The multilayer construct of either of paragraphs 1 or 2, wherein the UV resistant fluoropolymer adhesive is functionalized with one or more moieties selected from an olefinic moiety, a hydroxyl, an ether moiety, a methacrylate, an acrylate, an epoxide, a silane moiety, a phosphoric acid moiety, a sulfonic acid moiety or a carboxylic acid moiety.

4. The multilayer construct of paragraph 3, wherein UV resistant fluoropolymer adhesive is a tetrafluoroethylene functionalized with olefinic moieties and hydroxyl moieties.

5. The multilayer construct of paragraph 3, wherein the UV resistant fluoropolymer adhesive is a chlorotrifluoro ethylene polymer functionalized with alkyl vinyl ether moieties and hydroxyl moieties.

6. The multilayer construct of paragraph 3, wherein the UV resistant fluoropolymer adhesive is a polyperfluoroalkyl methacrylate or polyperfluoroalkyl acrylate.

7. The multilayer construct of paragraph 3, wherein the UV resistant fluoropolymer adhesive is a perfluorourethane alkyd, a perfluoropolyether, a perfluoro silane, or a perfluoro epoxide.

8. The multilayer construct of paragraph 3, wherein the UV resistant fluoropolymer adhesive is a functionalized perfluoro-polyether polymer functionalized with an alkyl amide, an alkoxy silane or a phosphate moiety.

9. The multilayer construct of paragraph 3, wherein the UV resistant fluoropolymer adhesive is an amorphous fluoropolymer.

10. The multilayer construct of any of paragraphs 1 through 9, wherein the third layer is selected from a fluoropolymers, polyethylenes, polypropylenes, nylons, EPDM, polyesters, polycarbonates, ethylene-propylene elastomer copolymers, polystyrenes, ethylene-styrene copolymers, terpolymers of ethylene-styrene and other C3-C20 olefins, copolymers of ethylene or propylene with acrylic or methacrylic acids, polyacrylates, polymethacrylates, ethylene-propylene copolymers, ethyl vinyl acetate (EVA), ethylene butyl acrylate (EBA) ethylene methyl acrylate (EMA), ionomers, acid functionalized polyolefins, polyurethanes, olefin elastomers, thermoplastic silicones, polyvinyl butyrals or mixtures thereof.

11. The multilayer construct of any of paragraphs 1 through 10, wherein the multilayer construct has a visible light transmission of at least 85%.

12. The multilayer construct of any of paragraphs 1 through 10, wherein the visible light transmission is at least 90%.

13. The multilayer construct of any of paragraphs 1 through 10, wherein the visible light transmission is at least 92%.

14. The multilayer construct of any of paragraphs 1 through 10, wherein the UV resistant fluoropolymer adhesive further comprises a UV absorber.

15. The multilayer construct of paragraph 14, wherein the UV absorber is a benzophenone, a benzotriazole, a triazine, or a benzoxazinone.

16. The multilayer construct of any of paragraphs 1 through 15, wherein the third layer comprises a light stabilizer.

17. The multilayer construct of paragraph 16, wherein the light stabilizer is a hindered amine light stabilizer.

18. The multilayer construct of any of paragraphs 1 through 17, further comprising a crosslinker or a crosslinking agent.

19. The multilayer construct of paragraph 18, wherein the crosslinker is selected from a mono, di or multifunctional containing agent containing amines, amides, anhydrides, phenolics, isocyanates, hydroxyls, carboxyls, titanates, zirconates, aluminates, proton donors, peroxides, silanes, metal ions, moisture or combinations thereof.

20. The multilayer construct of paragraph 18, wherein the crosslinking agent is selected from heat, pressure, moisture, pH change, UV light, thermal initiators, such as peroxides, persulfates, light sensitive initiators, gamma rays, electron beams, acid catalysts, base catalysts, metal ions or combinations thereof.

21. A front sheet or a back sheet construct of an electronic device comprising a multilayer construct of any of paragraphs 1 through 20.

22. A medical or pharmaceutical packaging construct comprising a multilayer construct of any of paragraphs 1 through 20.

23. The medical or pharmaceutical packaging construct of paragraph 22, wherein the fluoropolymer adhesive layer is essentially free of UV absorbers.

24. A method to sterilize a packaged medical or pharmaceutical component comprising the packaging construct of paragraph 23 by irradiating the packaged component with UV radiation through the packaging construct.

25. A construct comprising: a fluoropolymer first layer; and a UV resistant fluoropolymer adhesive layer.

26. The multilayer construct of paragraph 25, wherein the fluoropolymer first layer is selected from poly vinylidene fluoride (PVDF), polyvinyl fluoride (PVF), polytetrafluoroethylene (PTFE), a copolymer of tetrafluoroethylene and perfluoro methylvinylether (PFA), ethylene tetrafluoroethylene copolymer (ETFE), polychlorotrifluoroethylene (PCTFE), ethylene chlorotrifluoroethylene copolymer (ECTFE), fluorinated ethylene propylene copolymer (FEP), a copolymer of ethylene and fluorinated ethylene propylene (EFEP), a terpolymer of tetrafluoroethylene, hexafluoropropylene, and vinylidene fluoride (THV), a terpolymer of tetrafluoroethylene, hexafluoropropylene, and ethylene (HTE), or any combination thereof.

27. The multilayer construct of either of paragraphs 25 or 26, wherein the UV resistant fluoropolymer adhesive is functionalized with one or more moieties selected from an olefinic moiety, a hydroxyl, an ether moiety, a methacrylate, an acrylate, an epoxide, a silane moiety, a phosphoric acid moiety, a sulfonic acid moiety or a carboxylic acid moiety.

28. The multilayer construct of paragraph 27, wherein the UV resistant fluoropolymer adhesive is a tetrafluoroethylene functionalized with olefinic moieties and hydroxyl moieties.

29. The multilayer construct of paragraph 27, wherein the UV resistant fluoropolymer adhesive is a chlorotrifluoroethylene polymer functionalized with alkyl vinyl ether moieties and hydroxyl moieties.

30. The multilayer construct of paragraph 27, wherein the UV resistant fluoropolymer adhesive is a polyperfluoroalkyl methacrylate or polyperfluoroalkyl acrylate.

31. The multilayer construct of paragraph 27, wherein the UV resistant fluoropolymer adhesive is a perfluorourethane alkyd, a perfluoropolyether, a perfluoro silane, or a perfluoro epoxide.

32. The multilayer construct of paragraph 27, wherein the UV resistant fluoropolymer adhesive is a functionalized perfluoro-polyether polymer functionalized with an alkyl amide, an alkoxy silane or a phosphate moiety.

33. The multilayer construct of paragraph 27, wherein the UV resistant fluoropolymer adhesive is an amorphous fluoropolymer.

34. The multilayer construct of any of paragraphs 25 through 33, wherein the multilayer construct has a visible light transmission of at least 85%.

35. The multilayer construct of any of paragraphs 25 through 33, wherein the visible light transmission is at least 90%.

36. The multilayer construct of any of paragraphs 25 through 33, wherein the visible light transmission is at least 92%.

37. The multilayer construct of any of paragraphs 25 through 33, wherein the UV resistant fluoropolymer adhesive further comprises a UV absorber.

38. The multilayer construct of paragraph 37, wherein the UV absorber is a benzophenone, a benzotriazole, a triazine, or a benzoxazinone.

39. The multilayer construct of any of paragraphs 25 through 38, further comprising a crosslinker or a crosslinking agent.

40. The multilayer construct of paragraph 39, wherein the crosslinker is selected from a mono, di or multifunctional containing agent containing amines, amides, anhydrides, phenolics, isocyanates, hydroxyls, carboxyls, titanates, zirconates, aluminates, proton donors, peroxides, silanes, metal ions, moisture or combinations thereof.

41. The multilayer construct of paragraph 39, wherein the crosslinking agent is selected from heat, pressure, moisture, pH change, UV light, thermal initiators, such as peroxides, persulfates, light sensitive initiators, gamma rays, electron beams, acid catalysts, base catalysts, metal ions or combinations thereof.

42. A front sheet or back sheet construct of an electronic device comprising a multilayer construct of any of paragraphs 25 through 41.

43. A medical or pharmaceutical packaging construct comprising a multilayer construct of any of paragraphs 25 through 41.

44. The medical or pharmaceutical packaging construct of paragraph 43, wherein the UV resistant fluoropolymer adhesive layer is essentially free of UV absorbers.

45. A method to sterilize a packaged medical or pharmaceutical component comprising the packaging construct of paragraph 44 by irradiating the packaged component with UV radiation through the packaging construct.

46. A method to prepare a multilayer construct, comprising the step:
applying a UV resistant fluoropolymer adhesive to a fluoropolymer first layer to provide a multilayer construct.

47. The method of paragraph 46, wherein the fluoropolymer first layer is selected from poly vinylidene fluoride (PVDF), polyvinyl fluoride (PVF), polytetrafluoroethylene (PTFE), a copolymer of tetrafluoroethylene and perfluoro methylvinylether (PFA), ethylene tetrafluoroethylene copolymer (ETFE), polychlorotrifluoroethylene (PCTFE), ethylene chlorotrifluoroethylene copolymer (ECTFE), fluorinated ethylene propylene copolymer (FEP), a copolymer of ethylene and fluorinated ethylene propylene (EFEP), a terpolymer of tetrafluoroethylene, hexafluoropropylene, and vinylidene fluoride (THV), a terpolymer of tetrafluoroethylene, hexafluoropropylene, and ethylene (HTE), or any combination thereof.

48. The method of either of paragraphs 46 or 47, wherein the UV resistant fluoropolymer adhesive is functionalized with one or more moieties selected from an olefinic moiety, a hydroxyl, an ether moiety, a methacrylate, an acrylate, an epoxide, a silane moiety, a phosphoric acid moiety, a sulfonic acid moiety or a carboxylic acid moiety.

49. The method of paragraph 48, wherein the UV resistant fluoropolymer adhesive is a tetrafluoroethylene functionalized with olefinic moieties and hydroxyl moieties.

50. The method of paragraph 48, wherein the UV resistant fluoropolymer adhesive is a chlorotrifluoroethylene polymer functionalized with alkyl vinyl ether moieties and hydroxyl moieties.

51. The method of paragraph 48, wherein the UV resistant fluoropolymer adhesive is a polyperfluoroalkyl methacrylate or polyperfluoroalkyl acrylate.

52. The method of paragraph 48, wherein the UV resistant fluoropolymer adhesive is a perfluorourethane alkyd, a perfluoropolyether, a perfluoro silane, or a perfluoro epoxide.

53. The method of paragraph 48, wherein the UV resistant fluoropolymer adhesive is a functionalized perfluoro-polyether polymer functionalized with an alkyl amide, an alkoxy silane or a phosphate moiety.

54. The method of paragraph 48, wherein the UV resistant fluoropolymer adhesive is an amorphous fluoropolymer.

55. The method of any of paragraphs 46 through 54, wherein the multilayer construct has a visible light transmission of at least 85%.

56. The method of any of paragraphs 46 through 54, wherein the visible light transmission is at least 90%.

57. The method of any of paragraphs 46 through 54, wherein the visible light transmission is at least 92%.

58. The method of any of paragraphs 46 through 54, wherein the UV resistant fluoropolymer adhesive further comprises a UV absorber.

59. The method of paragraph 58, wherein the UV absorber is a benzophenone, a benzotriazole, a triazine, or a benzoxazinone.

60. The method of any of paragraphs 46 through 59, further comprising admixing a crosslinker or a crosslinking agent with the UV resistant fluoropolymer adhesive.

61. The method of paragraph 60, wherein the crosslinker is selected from a mono, di or multifunctional containing agent containing amines, amides, anhydrides, phenolics, isocyanates, hydroxyls, carboxyls, titanates, zirconates, aluminates, proton donors, peroxides, silanes, metal ions, moisture or combinations thereof.

62. The method of paragraph 60, wherein the crosslinking agent is selected from heat, pressure, moisture, pH change, UV light, thermal initiators, such as peroxides, persulfates, light sensitive initiators, gamma rays, electron beams, acid catalysts, base catalysts, metal ions or combinations thereof.

63. A packaged article, comprising a construct of any of paragraphs 25 through 41 and instructions to apply the construct to a third layer.

64. A packaged article, comprising a construct of any of paragraphs 25 through 41 contained in a vacuum sealed container or package.

### EXAMPLES

### Example 1

The following example further illustrates the concept of this invention. A S-layer clear laminate was constructed of an ETFE film as the outer weatherable layer with a fluoropolymer adhesive based on Daikin Zeffle® adhesive, and a PET film. The Zeffle® adhesive can be used as received or further formulated with one or more UV absorbers to block UV radiation from reaching the PET film underneath if the PET is not pre-formulated with UV additives. As an example, 48 g of Zeffle GK-570 adhesive solution (Daikin America) was diluted with 25 g of ethyl acetate solvent, followed by the addition of 5.4 g of Cyasorb UV 5411 (Cytech Industries). The mixture was mixed thoroughly with a high speed mixer. 6.5 g of isocyanate crosslinker, Desumodur N3300 (Bayer,) was added to the diluted Zeffle solution, and again mixed thoroughly with a high speed mixer to afford a final adhesive solution. The final adhesive solution was optically clear.

The formulation was applied to a clear 10 mil thick polyester (PET) film (SG00 from SKC of Korea) using a square drawdown bar to obtain a wet film thickness of approximately 2.5 mil. The coated polyester film was placed in an oven set at 90° C. for around 15 minutes to dry off the solvent. The targeted dry film thickness was 0.5 mil. The dried Zeffle coated polyester film was heat laminated (at 120° C.) to a clear 1 mil thick ETFE film (Norton® ETFE film from Saint-Gobain Performance Plastics Corp.). The finished laminate was tested for the interlaminar peel strength (90 degree T-pull) and light transmission testing (visible light range between 400 and 1,100 nm). The measured peel strength was 5.7 N/cm. The % light transmission was estimated to be >85% based on the preliminary study of PET film coated with Zeffle adhesive. This demonstrates that the ETFE-Zeffle adhesive-PET laminate would be an optically clear laminate structure.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

In the specification and in the claims, the terms “including” and “comprising” are open-ended terms and should be interpreted to mean “including, but not limited to . . . .” These terms encompass the more restrictive terms “consisting essentially of” and “consisting of:”

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Film thicknesses are set forth herein in terms of “mils”, wherein one mil is equal to 0.001 inch.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

What is claimed is:

1. A multilayer construct comprising:
a fluoropolymer first layer;
a UV resistant fluoropolymer adhesive layer, and
a third layer, wherein the fluoropolymer adhesive layer is between the first and third layers.

2. The multilayer construct of claim 1, wherein the fluoropolymer first layer is selected from poly vinylidene fluoride (PVDF), polyvinyl fluoride (PVF), polytetrafluoroethylene (PTFE), a copolymer of tetrafluoroethylene and perfluoro methylvinylether (PFA), ethylene tetrafluoroethylene copolymer (ETFE), polychlorotrifluoroethylene (PCTFE), ethylene chlorotrifluoroethylene copolymer (ECTFE), fluorinated ethylene propylene copolymer (FEP), a copolymer of ethylene and fluorinated ethylene propylene (EFEP), a terpolymer of tetrafluoroethylene, hexafluoropropylene, and vinylidene fluoride (THV), a terpolymer of tetrafluoroethylene, hexafluoropropylene, and ethylene (HTE), or any combination thereof.

3. The multilayer construct of claim 1, wherein the UV resistant fluoropolymer adhesive is functionalized with one or more moieties selected from an olefinic moiety, a hydroxyl, an ether moiety, a methacrylate, an acrylate, an epoxide, a silane moiety, a phosphoric acid moiety, a sulfonic acid moiety or a carboxylic acid moiety.

4. The multilayer construct of claim 3, wherein UV resistant fluoropolymer adhesive is a tetrafluoroethylene functionalized with olefinic moieties and hydroxyl moieties or a chlorotrifluoroethylene polymer functionalized with alkyl vinyl ether moieties and hydroxyl moieties.

5. The multilayer construct of claim 1, wherein the third layer is selected from a fluoropolymers, polyethylenes, polypropylenes, nylons, EPDM, polyesters, polycarbonates, ethylene-propylene elastomer copolymers, polystyrenes, ethylene-styrene copolymers, terpolymers of ethylene-styrene and other C3-C20 olefins, copolymers of ethylene or propylene with acrylic or methacrylic acids, polyacrylates, polymethacrylates, ethylene-propylene copolymers, ethyl vinyl acetate (EVA), ethylene butyl acrylate (EBA) ethylene methyl acrylate (EMA), ionomers, acid functionalized polyolefins, polyurethanes, olefin elastomers, thermoplastic silicones, polyvinyl butyrals or mixtures thereof.

6. The multilayer construct of claim 1, wherein the multilayer construct has a visible light transmission of at least 85%.

7. The multilayer construct of claim 1, wherein the UV resistant fluoropolymer adhesive further comprises a UV absorber.

8. The multilayer construct of claim 1, further comprising a crosslinker or a crosslinking agent in the UV resistant fluoropolymer adhesive layer.

9. A front sheet of an electronic device, a back sheet of an electronic device, a medical packaging construct or a pharmaceutical packaging construct comprising the multilayer construct of claim 1.

10. The medical or pharmaceutical packaging construct of claim 9, wherein the UV resistant fluoropolymer adhesive layer is essentially free of additional UV absorbers.

11. A method to sterilize a packaged medical or pharmaceutical component comprising the packaging construct of claim 23 by irradiating the packaged component with UV radiation through the packaging construct.

12. A construct comprising:
a fluoropolymer first layer; and
a UV resistant fluoropolymer adhesive layer.

13. The multilayer construct of claim 12, wherein the fluoropolymer first layer is selected from poly vinylidene fluoride (PVDF), polyvinyl fluoride (PVF), polytetrafluoroethylene (PTFE), a copolymer of tetrafluoroethylene and perfluoro methylvinylether (PFA), ethylene tetrafluoroethylene copolymer (ETFE), polychlorotrifluoroethylene (PCTFE), ethylene chlorotrifluoroethylene copolymer (ECTFE), fluorinated ethylene propylene copolymer (FEP), a copolymer of ethylene and fluorinated ethylene propylene (EFEP), a terpolymer of tetrafluoroethylene, hexafluoropropylene, and vinylidene fluoride (THV), a terpolymer of tetrafluoroethylene, hexafluoropropylene, and ethylene (HTE), or any combination thereof.

14. The multilayer construct of claim 12, wherein the UV resistant fluoropolymer adhesive is functionalized with one or more moieties selected from an olefinic moiety, a hydroxyl, an ether moiety, a methacrylate, an acrylate, an epoxide, a silane moiety, a phosphoric acid moiety, a sulfonic acid moiety or a carboxylic acid moiety.

15. The construct of claim 14, wherein the UV resistant fluoropolymer adhesive is a tetrafluoroethylene functionalized with olefinic moieties and hydroxyl moieties or a chlorotrifluoroethylene polymer functionalized with alkyl vinyl ether moieties and hydroxyl moieties.

16. The multilayer construct of claim 12, wherein the multilayer construct has a visible light transmission of at least 85%.

17. The multilayer construct of claim 12, wherein the UV resistant fluoropolymer adhesive further comprises a UV absorber.

18. The multilayer construct of claim 12, further comprising a crosslinker or a crosslinking agent.

19. A front sheet or back sheet construct of an electronic device comprising a multilayer construct of claim 12.

20. A method to prepare a multilayer construct, comprising the step:
applying a UV resistant fluoropolymer adhesive to a fluoropolymer first layer to provide a multilayer construct.

21. The method of claim 20, wherein the fluoropolymer first layer is selected from poly vinylidene fluoride (PVDF), polyvinyl fluoride (PVF), polytetrafluoroethylene (PTFE), a copolymer of tetrafluoroethylene and perfluoro methylvinylether (PFA), ethylene tetrafluoroethylene copolymer (ETFE), polychlorotrifluoroethylene (PCTFE), ethylene chlorotrifluoroethylene copolymer (ECTFE), fluorinated ethylene propylene copolymer (FEP), a copolymer of ethylene and fluorinated ethylene propylene (EFEP), a terpolymer of tetrafluoroethylene, hexafluoropropylene, and vinylidene fluoride (THY), a terpolymer of tetrafluoroethylene, hexafluoropropylene, and ethylene (HTE), or any combination thereof.

22. The method of claim 20, wherein the UV resistant fluoropolymer adhesive is functionalized with one or more moieties selected from an olefinic moiety, a hydroxyl, an ether moiety, a methacrylate, an acrylate, an epoxide, a silane moiety, a phosphoric acid moiety, a sulfonic acid moiety or a carboxylic acid moiety.

23. The method of claim 22, wherein the UV resistant fluoropolymer adhesive is a tetrafluoroethylene functionalized with olefinic moieties and hydroxyl moieties or a chlorotrifluoroethylene polymer functionalized with alkyl vinyl ether moieties and hydroxyl moieties.

24. The method of claim 20, further comprising admixing a crosslinker or a crosslinking agent with the UV resistant fluoropolymer adhesive.
